(19) United States
(12) Patent Application Publication
(10) Pub. No.: US 2015/0320712 A1
(43) Pub. Date: Nov. 12, 2015

(54) Nutritional Composition for Gastrostomy-Tube Patients

(71) Applicant: AJINOMOTO CO., INC., Tokyo (JP)

(72) Inventors: Akira Horiuchi, Matsumoto-shi (JP); Manabu Suzuki, Tokyo (JP); Ryosei Sakai, Kawasaki-shi (JP)

(73) Assignee: AJINOMOTO CO., INC., Tokyo (JP)

(21) Appl. No.: 14/688,163

(22) Filed: Apr. 16, 2015

Related U.S. Application Data
(63) Continuation of application No. PCT/JP2013/078384, filed on Oct. 18, 2013.

(30) Foreign Application Priority Data
Oct. 19, 2012 (JP) 2012-232238

(51) Int. Cl. A61K 31/198; A61K 36/48; A61K 31/405; A61J 1/14; A61K 31/401; A61J 3/00; A61J 1/20; A61K 31/718; A61K 31/4172

(52) U.S. Cl. CPC A61K 31/198; A61K 31/718; A61K 36/48; A61K 31/405; A61K 31/4172; A61K 31/401; A61J 3/002; A61J 1/2093; A61J 1/1475

(57) ABSTRACT

A nutritional composition for gastrostomy-tube patients who are at an increased development risk of aspiration pneumonia and require careful risk management, which can be more safely consumed by gastrostomy-tube patients while suppressing the development of aspiration pneumonia, is administered to those patients who require risk management for aspiration pneumonia, and contains a protein source, a carbohydrate source, and a lipid source, the protein source including only amino acids.

This application is a Continuation of, and claims priority under 35 U.S.C. §120 to, International Application No. PCT/JP2013/078384, filed Oct. 18, 2013, and claims priority therethrough under 35 U.S.C. §119 to Japanese Patent Application No. 2012-232238, filed Oct. 19, 2012, the entireties of which are incorporated by reference herein.

### TECHNICAL FIELD

The present invention relates to a nutritional composition for gastrostomy-tube patients which is administered to those patients who require risk management for aspiration pneumonia, and a composition for preparing the nutritional composition for gastrostomy-tube patients which can be administered through a tube.

The present invention relates to a nutritional composition for gastrostomy-tube patients which is administered to those patients who require risk management for aspiration pneumonia, and a composition for preparing the nutritional composition for gastrostomy-tube patients which can be administered through a tube.

### BACKGROUND ART

Examples of a general method of supplying nutrition to a patient, such as a bedridden patient, who has difficulty with oral nutrition ingestion include a nasogastric tube feeding method in which a nutrient is administered to the stomach via a tube through the nose to the stomach or to the duodenum; a method of supplying nutrition through a gastric fistula in which a nutrient is directly administered to the stomach from a gastric fistula which is installed by making holes over the gastric wall and the abdominal wall; or a method of supplying nutrition through intestinal fistula in which a nutrient is directly administered to the intestines through an intestinal fistula which is installed by making holes over the intestinal wall and the abdominal wall of the duodenum or the jejunum. Particularly, in recent years, the method of supplying nutrition through a gastric fistula or the method of supplying nutrition through an intestinal fistula has rapidly become widespread due to development of percutaneous endoscopic gastrostomy (PEG) or percutaneous endoscopic jejunostomy (PEJ), in which a gastric fistula catheter or an intestinal fistula catheter can be installed with an endoscope without abdominal surgery.

In some cases, in the method of supplying nutrition through a gastric fistula, the gastric contents flow back to the esophagus, thereby causing development of aspiration pneumonia (pneumonia which develops by pulmonary aspiration or accidental ingestion). Particularly, there is a problem in that the risk of developing aspiration pneumonia is high for a bedridden elderly person. For this reason, in a method of supplying nutrition, a nutritional composition is administered through an intestinal fistula in which the risk of developing aspiration pneumonia is low since it is possible to directly administer the nutritional composition to the intestines with respect to a patient who has a high risk of developing aspiration pneumonia.

However, the intestines have a narrow lumen and cannot store a nutrient therein like the stomach does. Therefore, there is a concern that an excessively high injection rate may cause diarrhea or glucose level abnormality such as dumping syndrome. Therefore, in the method of supplying nutrition through an intestinal fistula, it is necessary to control dosing at a constant rate using a pump for enteral nutrition. In addition, in the intestinal fistula catheter, it is necessary to remove any blockages of the catheter at a higher frequency compared to the case of the gastric fistula since the intestinal fistula catheter is narrower and longer than the intestinal fistula. In this manner, the method of supplying nutrition through an intestinal fistula requires complicated management in the hospital or at home while the risk of developing aspiration pneumonia is suppressed. In addition, a more advanced technique is required for installing an intestinal fistula compared to a gastric fistula. Therefore, it is desirable that nutrition management is performed through the method of supplying nutrition through a gastric fistula even for patients who have high risk of developing aspiration pneumonia and require sufficient risk management.

In the method of supplying nutrition through a gastric fistula, it is considered that a semisolid nutrient or a highly viscous nutrient rarely flows back to the esophagus from the stomach and rarely induces diarrhea, unlike a liquid nutrient. For example, JP 2011-50278 discloses a fluid diet consisting of a nutritional composition and a thickener, which has fluidity in a liquid form or a fluid form until it is administered to the stomach. Once both the nutritional composition and the thickener are mixed with each other, a semi-solidified form occurs, that is,the fluidity decreases or is lost, inside the stomach after being administered to the stomach. It is regarded that the fluid diet is easily administered through a tube since the fluidity is high until being administered to the stomach, and can suppress flowing back to the esophagus from the stomach, diarrhea, or the like, by being semi-solidified inside the stomach. In addition, JP2007-137792 discloses an enteral nutrient for a gastric fistula which can maintain adequate viscosity of greater than or equal to 200 mPa·s (27° C.) when the enteral nutrient is diluted by gastric secretions, using acetylated adipic acid cross-linked starch as a viscosity modifier. The enteral nutrient for a gastric fistula also can maintain adequate viscosity inside the stomach, and therefore, it is regarded that it is possible to suppress vomiting due to gastroesophageal reflux or diarrhea due to dyspepsia caused by insufficient retention time of the nutrient.

It is considered that it is important to promptly discharge the contents from the stomach to the duodenum in order to suppress aspiration pneumonia. For example, Yoshihisa URITA, et al., (Journal of Smooth Muscle Research, 2002, vol. 6, J-121 to J-127), reports that the gastric emptying rates of a liquid diet with a high fat content and a liquid diet with a low fat content are almost the same when the total calories are substantially the same, and that the discharge rate of the liquid diet from the stomach becomes lower as the total calories of the liquid becomes higher regardless of the level of the fat content.

In this manner, in the related art, it is regarded that a nutrient with high viscosity or in a semisolid form is preferable over a liquid nutrient in order to reduce the risk of developing aspiration pneumonia in the method of supplying nutrition through a gastric fistula. In addition, it is considered that the discharge rate of the contents from the stomach to the duodenum depends more on the total calories than the composition of the nutrient.

In addition, as a nutritional composition for gastrostomy-tube patients which is used for nutrition supply through a gastric fistula, it is preferable that the composition be a solid composition in a powdered form, a granular form, or the like, and be prepared in a liquid composition which can be administered through a tube by being mixed with water or the like at the time of administration to a patient, in terms of favorable long-term storage stability. In general, the nutritional liquid composition for gastrostomy-tube patients is prepared by mixing a nutritional powder-like composition for gastrostomy-tube patients with room-temperature water or tepid water, in a container, for administration through a gastric fistula, which has a pouring spout which is connected to a tube. The prepared nutritional liquid composition is administered to a patient from a gastric fistula through the tube which is connected to the pouring spout of the container for administration through a gastric fistula. The upper portion of a general container for administration through a gastric fistula is open and the lower portion thereof is provided with a pouring spout which allows communication with the tube that is connected to the gastric fistula. For this reason, there are problems in that falling bacteria or the like are mixed in through an opening of the upper portion of the container for administration through a gastric fistula at the time of preparing the nutritional composition for gastrostomy-tube patients or administering the nutritional composition to the gastric fistula through a tube, and there is a high risk of causing bacterial infection.

A nutritional composition in a container is commercially available in which a sealed container is aseptically filled with a nutritional liquid composition which can be administered through a tube, in advance. Since this nutritional composition in a container can be directly administered to the gastric fistula from the container through a tube, preparation in use is unnecessary. Therefore, there is an advantage in that the risk of bacterial infection is low. However, there is a problem in that the storage period at room temperature is short.

### CITATION LIST

### Patent Literature

[PTL1] JP 2011-50278 A

[PTL2] JP2007-137792 A

[NPTL1] Yoshihisa URITA, et al., Journal of Smooth Muscle Research, 2002, vol. 6, J-121 to J-127

### SUMMARY OF THE INVENTION

### Technical Problem

An object of the present invention is to provide a nutritional composition, for gastrostomy-tube patients who are at an increased development risk of aspiration pneumonia and require careful risk management, which can be more safely consumed by the gastrostomy-tube patients while suppressing the development of aspiration pneumonia.

Furthermore, another object of the present invention is to provide a composition in a container for preparing the nutritional composition which is prepared in a liquid form by being mixed with a liquid such as water when in use and is used for nutrition supply through a gastric fistula, in which it is possible to perform preparation in use within a sealed container, and the risk of bacterial infection due to falling bacteria or the like when administering the composition to a patient is significantly decreased.

### Solution to Problem

The present inventors have completed the present invention by conducting extensive studies in order to solve the above-described problems, and as a result, they have found that the speed of discharging a nutritional composition, which contains only amino acids as a protein source, from the stomach is higher than that of a nutritional composition which contains proteins, peptides, or the like, unlike the common knowledge in the related art in that the risk of developing aspiration pneumonia in a semisolid nutrient is lower than that in a liquid nutrient; and the development of aspiration pneumonia can be suppressed.

That is, the present invention provides a nutritional composition for gastrostomy-tube patients in the following (1) to (6) and a device useful for preparing the nutritional composition for gastrostomy-tube patients which can be administered through a tube in (7) to (19).

(1) A nutritional composition for gastrostomy-tube patients,

in which the composition is administered to those patients who require risk management for aspiration pneumonia,

in which the composition comprises a protein source, a carbohydrate source, and a lipid source, and

in which the protein source consists essentially of amino acids.

(2) The nutritional composition for gastrostomy-tube patients according to (1), in which protein source is, per dry weight:

0.2 W/W % to 1.5 W/W % of L-isoleucine,

0.5 W/W % to 2.0 W/W % of L-leucine,

0.5 W/W % to 2.0 W/W % of L-lysine,

0.2 W/W % to 1.5 W/W % of L-methionine,

0.5 W/W % to 2.0 W/W % of L-phenylalanine,

0.2 W/W % to 1.5 W/W % of L-threonine,

0.05 W/W % to 0.5 W/W % of L-tryptophan,

0.2 W/W % to 1.5 W/W % of L-valine,

0.5 W/W % to 2.0 W/W % of L-histidine,

0.5 W/W % to 2.5 W/W % of L-arginine,

0.5 W/W % to 2.0 W/W % of L-alanine,

1.0 W/W % to 4.0 W/W % of L-aspartic acid,

1.0 W/W % to 4.0 W/W % of L-glutamine,

0.2 W/W % to 1.5 W/W % of glycine,

0.2 W/W % to 1.5 W/W % of L-proline,

0.5 W/W % to 2.5 W/W % of L-serine, and

0.05 W/W % to 0.5 W/W % of L-tyrosine.

(3) The nutritional composition for gastrostomy-tube patients according to (1) or (2),

in which the carbohydrate source is 70 W/W % to 85 W/W % of dextrin per dry weight, and

wherein the lipid source is 0.1 W/W % to 10 W/W % of soybean oil per dry weight.

(4) The nutritional composition for gastrostomy-tube patients according to any one of (1) to (3),

wherein the lipid source is 0.3 W/W % to 1.0 W/W % per dry weight.

(5) The nutritional composition for gastrostomy-tube patients according to any one of (1) to (4),

in which the composition is administered such that the dose per day is greater than or equal to 900 kcal.

(6) The nutritional composition for gastrostomy-tube patients according to any one of (1) to (5),

in which the composition can be continuously administered over 20 months or longer while suppressing the incidence rate of aspiration pneumonia to be less than or equal to 3%.

(7) A device useful for preparing a nutritional composition for gastrostomy-tube patients, the device comprising:

a composition comprising at least one source selected from the group consisting of a protein source, a carbohydrate source, and a lipid source;,

a multi-chamber container having at least two chambers and a communicating partition wall portion formed of a flexible film partitioning the at least two chambers, the composition being stored in said container;

wherein one of the at least two chambers is a water injection chamber and has a storage capacity greater than or equal to 200 mL of a liquid, and further comprises a resealable water injection port through which liquid can be injecedt and dischargeed a liquid; and

wherein at least one of the at least two chambers comprises at least one medicine storage chamber, in which at least a part of said composition is stored; and

in which in at least one of the medicine storage chambers, at least a part of the composition is stored in a solid form.

(8) The device according to (7),

wherein the partition wall portion is configured and arranged to allow communication between the water injection chamber and at least one medicine storage chamber, such that the nutritional composition for gastrostomy-tube patients can be prepared in the sealed multi-chamber container by pressing the water injection chamber from outside the chamber when the water injection port is sealed after injecting water into the water injection chamber through the water injection port.

(9) The device according to (7) or (8),

in which the multi-chamber container further comprises a discharge port comfigured and arranged to allow communication with a tube that is connectable to a gastric fistula.

(10) The device according to any one of (7) to (9), wherein

one of the at least two chambers is a first medicine storage chamber in which a part of said composition is stored in a solid form, and a second of the at least two chambers is a second medicine storage chamber in which the remainder of the composition is stored; and

wherein at least a carbohydrate source is stored in the first medicine storage chamber.

(11) The device according to (10),

in which L-glutamine is stored in the first medicine storage chamber.

(12) The device according to (10) or (11),

wherein at least one composition selected from the group consisting of a fat emulsion, an antiflatulent, and a medicine for treating a disease, are stored in the second medicine storage chamber.

(13) The device according to any one of (7) to (12), wherein

the multi-chamber container further comprises a pipe member having a cylindrical space and a rubber plug that blocks the cylindrical space,

the cylindrical space allows communication between the multi-chamber container and outside the device, and

the rubber plug can be penetrated by a hollow needle and can be resealed by withdrawing the hollow needle.

(14) The device according to any one of (7) to (13), further comprising an inert gas in the water injection chamber.

(15) The device according to any one of (7) to (14),

in which the multi-chamber container is sterilized by radiation.

(16) The device according to any one of (7) to (15), further comprising:

an external packaging bag and a deoxygenating agent, a drying agent, or both, therein;

wherein the multi-chamber container is air-tightly stored in said external packaging bag.

(17) The device according to (16),

wherein the external packaging bag is formed of a flexible film which has light-shielding properties and through which gas does not permeate.

(18) The device according to any one of (7) to (17),

wherein the nutritional composition for gastrostomy-tube patients contains only amino acids as the protein source.

(19) The device useful for preparing a nutritional composition for gastrostomy-tube patients, the device comprising:,

a nutritional composition for gastrostomy-tube patients according to any one of (1) to (6);

a multi-chamber container having at least two chambers and a communicating partition wall portion formed of a flexible film partitioning the at least two chambers, the composition being stored in said container;

wherein one of the at least two chambers is a water injection chamber and has a storage capacity greater than or equal to 200 mL of a liquid, and further comprises a resealable water injection port through which liquid can be injected and discharged;

wherein at least one of the at least two chambers comprises at least one medicine storage chamber, in which at least a part of said composition is stored in a solid form.

(20) A method of manufacturing a device useful for preparing a nutritional composition for gastrostomy-tube patients, the method comprising:

providing a flexible film, a fluid port device, and a composition according to claim 1;

positioning said composition adjacent to said flexible film;

positioning said fluid port device at an edge of said flexible film; and

sealing the flexible film to form a container having at least two compartments and a partition wall releasably separating the at least two compartments, said sealing including sealing said fluid port device at said edge such that the fluid port device fluidly communicates one of said compartments with the exterior of said container, and said composition is sealed in another of said at least two compartments.

### Effect of the Invention

The nutritional composition for gastrostomy-tube patients according to the present invention is promptly discharged from the stomach to the duodenum when it is injected through a gastric fistula. For this reason, it is possible to suppress the risk of developing aspiration pneumonia using the nutritional composition for gastrostomy-tube patients according to the present invention in a method of supplying nutrition through a gastric fistula. With this feature, the nutritional composition for gastrostomy-tube patients according to the present invention is favorable as a nutrient through a gastric fistula for gastrostomy-tube patients, such as an elderly person or a gastrostomy-tube patient who sleeps in a bed for greater than or equal to 50% of a day, who would easily develop aspiration pneumonia and require risk management. In particular, the nutritional composition for gastrostomy-tube patients according to the present invention is additionally favorable as a nutrient through a gastric fistula for gastrostomy-tube patients to whom nutrition is supplied through a gastric fistula over a long period of time.

In addition, at least a part of the composition for preparing the nutritional composition for gastrostomy-tube patients which can be administered through a tube according to the present invention is in a solid form, and therefore, the composition is suitable for long-term storage. Furthermore, with the composition for preparation, it is possible to perform preparation of the nutritional composition for gastrostomy-tube patients while in use within a sealed container, and the prepared nutritional liquid composition can be administered to a patient from the sealed container after the preparation while maintaining the sealed state except for when the container allows communication with a tube that is connected to a gastric fistula. For this reason, it is possible to markedly reduce the risk of bacterial infection due to falling bacteria or the like, using the composition for nutrition supply through a gastric fistula.

### BRIEF DESCRIPTION OF THE DRAWINGS

FIG. 1 is a view showing a mode of a multi-chamber container in which a composition for preparing a nutritional composition for gastrostomy-tube patients which can be administered through a tube according to the present invention is stored.

FIG. 2 is a view showing a mode of a multi-chamber container in which a composition for preparing a nutritional composition for gastrostomy-tube patients which can be administered through a tube according to the present invention is stored.

FIG. 3 is a view showing a mode of a multi-chamber container in which a composition for preparing a nutritional composition for gastrostomy-tube patients which can be administered through a tube according to the present invention is stored.

FIG. 4 is a view showing a mode of a multi-chamber container in which a composition for preparing a nutritional composition for gastrostomy-tube patients which can be administered through a tube according to the present invention is stored.

FIG. 5 is a view showing a curve in which a rate of ¹³CO₂ appearance in the breath ([% dose/h] value) at each point of time after administration of a labeled nutrient to a gastrostomy-tube patient is plotted, in Example 2.

FIG. 6 is a view showing a cumulative amount of ¹³CO₂ recovered in the breath at each point of time after the administration of the labeled nutrient which is calculated using FIG. 5, in Example 2.

FIG. 7 is a view showing a curve in which a rate of ¹³CO₂ appearance in the breath ([% dose/h] value) at each point of time after administration of a labeled nutrient to a healthy person is plotted, in Example 2.

FIG. 8 is a view showing a cumulative amount of ¹³CO₂ appearance in the breath at each point of time after the administration of the labeled nutrient which is calculated using FIG. 7, in Example 2.

### DETAILED DESCRIPTION OF THE INVENTION

### <Nutritional Composition for Gastrostomy-Tube Patients>

A nutritional composition for gastrostomy-tube patients (hereinafter, in some cases, referred to as a “nutritional composition according to the present invention”) according to the present invention is a nutritional composition which is injected through a gastric fistula; and is administered to those patients who require risk management for aspiration pneumonia. As shown in Example 2 herein, the gastric emptying rate of the nutritional composition according to the present invention is higher than that of a general semisolid enteral nutrition composition, and aspiration pneumonia rarely develops. For this reason, the nutritional composition according to the present invention is suitable for nutrition supply through a gastric fistula with respect to gastrostomy-tube patients who require risk management for aspiration pneumonia. For example, in a case where the nutritional composition for gastrostomy-tube patients according to the present invention is used as a primary nutrition supply source, that is,in a case where greater than or equal to 50% of calories required per day is taken from the nutritional composition for gastrostomy-tube patients according to the present invention, even in a case of gastrostomy-tube patients who are at an increased risk of aspiration pneumonia and require risk management, it is possible to continuously administer the composition to the patients over 5 months or longer, preferably 12 months or longer, and more preferably 20 months or longer. In addition, the nutritional composition for gastrostomy-tube patients according to the present invention can reduce the risk of developing aspiration pneumonia in the administration over a long period of time. For example, it is possible to continuously administer the composition to gastrostomy-tube patients over a long period of time, such as over 20 months or longer, while suppressing the risk of developing aspiration pneumonia to be less than or equal to 3% and preferably less than or equal to 1%.

The risk of aspiration pneumonia means a factor with high probability of developing aspiration pneumonia, and the gastrostomy-tube patients who require risk management for aspiration pneumonia means patients who require management in order to avoid or reduce the risk of developing aspiration pneumonia as much as possible.

Examples of the gastrostomy-tube patients who require risk management for aspiration pneumonia include gastrostomy-tube patients who sleep in a bed for greater than or equal to 50% of a day; gastrostomy-tube patients of 60 years of age or older; gastrostomy-tube patients who exhibit symptoms of vomiting, or who have decreased cough reflex; gastrostomy-tube patients who exhibit symptoms of diarrhea; gastrostomy-tube patients who are susceptible to infection; and gastrostomy-tube patients who have developed aspiration pneumonia in the past. Among them, the nutritional composition according to the present invention is preferably used for nutrition supply with respect to gastrostomy-tube patients who sleep in a bed for greater than or equal to 50% of a day, more preferably gastrostomy-tube patients who sleep in a bed for greater than or equal to 80% of a day, still more preferably gastrostomy-tube patients of 60 years of age or older who sleep in a bed for greater than or equal to 80% of a day, and still more preferably bedridden gastrostomy-tube patients of 60 years of age or older. Particularly, the nutritional composition according to the present invention is preferably used for nutrition supply with respect to patients who have decreased cough reflex in the bedridden gastrostomy-tube patients of 60 years of age or older.

In addition, the nutritional composition according to the present invention can also be used for nutrition supply to infants, in particular to premature babies or the like through a nasogastric tube feeding method, providing high gastric emptying rate and ease of digestion.

The nutritional composition according to the present invention contains only amino acids as a protein source. Since the nutritional composition according to the present invention does not contain proteins or peptides, it is almost unnecessary to digest the composition. For this reason, the content is promptly discharged from the stomach. Accordingly, the time for which the composition stays in the stomach can be shortened, and as a result, it is inferred that the development of aspiration pneumonia can be suppressed.

The amino acids are not particularly limited as long as the amino acids can be used for the purpose of supplying nutrition such as in a usual infusion solution or an enteral nutrient. However, crystalline amino acids are preferable. Each amino acid is not necessarily used as a free amino acid and may be used in forms of an inorganic acid salt, an organic acid salt, an ester form which is hydrolyzable within a living body, or the like.

All of D-forms, L-forms, or DL-forms may be used as the amino acids, but L-forms are preferable. Specific examples thereof include L-isoleucine, L-leucine, L-valine, L-lysine, L-methionine, L-phenylalanine, L-threonine, L-tryptophan, L-alanine, L-arginine, L-aspartic acid, L-cysteine, L-glutamic acid, L-histidine, L-proline, L-serine, L-tyrosine, and glycine. These amino acids can be used alone or a plurality of kinds thereof can be blended and combined. However, the combination of a plurality of kinds thereof is preferable, and among these, use of 8 kinds of essential amino acids including L-tryptophan, L-methionine, L-lysine, L-phenylalanine, L-leucine, L-isoleucine, L-valine, and L-threonine in combination is preferable. Moreover, use of the 8 kinds of essential amino acids and non-essential amino acids in combination is more preferable. Furthermore, blending in of branched amino acids including L-valine, L-isoleucine, and L-leucine is particularly preferable in terms of storage stability.

As the blending amount of each amino acid in the nutritional composition according to the present invention, the following are preferable, per dry weight.

L-isoleucine: 0.2 W/W % to 1.5 W/W %,

L-leucine: 0.5 W/W % to 2.0 W/W %,

L-lysine: 0.5 W/W % to 2.0 W/W %,

L-methionine: 0.2 W/W % to 1.5 W/W %,

L-phenylalanine: 0.5 W/W % to 2.0 W/W %,

L-threonine: 0.2 W/W % to 1.5 W/W %,

L-tryptophan: 0.05 W/W % to 0.5 W/W %,

L-valine: 0.2 W/W % to 1.5 W/W %,

L-histidine: 0.5 W/W % to 2.0 W/W %,

L-arginine: 0.5 W/W % to 2.5 W/W %,

L-alanine: 0.5 W/W % to 2.0 W/W %,

L-aspartic acid: 1.0 W/W % to 4.0 W/W %,

L-glutamine: 1.0 W/W % to 4.0 W/W %,

Glycine: 0.2 W/W % to 1.5 W/W %,

L-proline: 0.2 W/W % to 1.5 W/W %,

L-serine: 0.5 W/W % to 2.5 W/W %, and

L-tyrosine: 0.05 W/W % to 0.5 W/W %.

The nutritional composition according to the present invention contains a carbohydrate source. As the carbohydrate source, saccharides are preferable, and examples thereof include a monosaccharide, a disaccharide, and a polysaccharide. More specific examples thereof include glucose, fructose, mannose, galactose, sucrose, sugar, which may be purified sucrose, maltose, lactose, dextrin, maltodextrin, starch, corn starch, soybean oligosaccharides, and sugar alcohols. Two or more kinds of saccharides thereof may be blended in. As the carbohydrate source in the nutritional composition according to the present invention, dextrin is preferable.

The nutritional composition according to the present invention contains a lipid source. The lipid source is not particularly limited, but vegetable oil and animal oil are preferable. Examples of the vegetable oil include soybean oil, perilla oil, and corn oil, and soybean oil is preferable. In addition, perilla oil may be contained therein as vegetable oil which contains a large number of ω-3 based fatty acids. As the animal oil, fish oil which contains ω-3 based fatty acids such as eicosapentaenoic acid and decosahexaenoic acid is preferable. Two or more kinds of the above-described lipids may be blended into the composition. Particularly, the composition more preferably contains vegetable and/or animal oils which contains at least one of ω-3 based fatty acids selected from the group consisting of a-linolenic acid, eicosapentaenoic acid, and docosahexaenoic acid, and still more preferably contains soybean oil.

As the contents of the protein source, the carbohydrate source, and the lipid source in the nutritional composition according to the present invention, the content of the protein source is preferably 15 W/W % to 20 W/W %, the content of the carbohydrate source is preferably 75 W/W % to 85 W/W %, and the content of the lipid source is preferably 0.3 W/W % to 1 W/W %.

Additives such as vitamins and minerals which are usually used can be further blended with the nutritional composition according to the present invention. Examples of the vitamins include vitamin A, a vitamin B group, vitamin C, vitamin D, vitamin E, nicotinamide, folic acid, pantothenic acid, biotin, and choline. Examples of the minerals include sodium chloride, potassium chloride, calcium glycerophosphate, magnesium sulfate, manganese sulfate, zinc sulfate, ferrous sulfate, and copper sulfate. Furthermore, flavors, sweeteners, colorants, stabilizers, preservatives, pH adjusting agents or the like can be used as necessary.

More specific examples of the composition include “Elental (registered trade name)” which has a composition shown in Table 1 and is commercially available as an enteral nutrient.

TABLE 1

| | | Content in 100 g of | |
|---|---|---|---|
| | Constituent name | nutritional composition | |
| Amino acid | L-Isoleucine | 803 | mg |
| | L-Leucine | 1,124 | mg |
| | L-Lysine hydrochloride | 1,110 | mg |
| | L-Methionine | 810 | mg |
| | L-Phenylalanine | 1,089 | mg |
| | L-Threonine | 654 | mg |
| | L-Tryptophan | 189 | mg |
| | L-Valine | 876 | mg |
| | L-Histidine hydrochloride | 626 | mg |
| | hydrate | | |
| | L-Arginine hydrochloride | 1,406 | mg |
| | L-Alanine | 1,124 | mg |
| | Magnesium/potassium L-aspartic | 1,295 | mg |
| | acid | | |
| | Sodium L-aspartate monohydrate | 1,084 | mg |
| | L-Glutamine | 2,415 | mg |
| | Glycine | 631 | mg |
| | L-Proline | 788 | mg |
| | L-Serine | 1,449 | mg |
| | L-Tyrosine | 138 | mg |
| Carboh- | Dextrin | 79.26 | g |
| ydrate | | | |
| Lipid | Soybean oil | 636 | mg |
| Minerals | Sodium citrate hydrate | 770 | mg |
| | Potassium chloride | 188 | mg |
| | Calcium glycerophosphate | 1,031 | mg |
| | Ferrous gluconate dihydrate | 19.4 | mg |
| | Zinc sulfate hydrate | 9.85 | mg |
| | Manganese sulfate pentahydrate | 1. 63 | mg |
| | Copper sulfate | 1.03 | mg |
| | Potassium iodide | 24.5 | μg |
| Vitamins | Thiamine chloride hydrochloride | 242 | μg |
| | Riboflavin sodium phosphate | 320 | μg |
| | Pyridoxine hydrochloride | 334 | μg |
| | Cyanocobalamin | 0.9 | μg |
| | Calcium pantothenate | 1.49 | mg |
| | Nicotinamide | 2.75 | mg |
| | Folic acid | 55 | μg |
| | Biotin | 49 | μg |
| | Choline Bitartrate | 22.41 | mg |
| | Ascorbic acid | 9.75 | mg |
| | Retinol acetate | 810 | IU |
| | Tocopherol acetate | 4.13 | mg |
| | Ergocalciferol | 1.6 | μg |
| | Phytonadione | 11 | μg |

Additives (potassium sorbate, polysorbate 80, aspartame (L-phenylalanine compound), flavoring, soy lecithin, citric acid hydrate, lactose hydrate, and carmellose sodium)

The nutritional composition according to the present invention may contain a component which is administered to a patient for the purpose other than for the supply of nutrition. Examples of the component include an antiflatulent or a medicine which is administered for the purpose of treating a specific disease. Examples of the medicine for treating a disease include antibiotics, antipyretic analgesic antiphlogistics, and anti-tumor agents.

The nutritional composition according to the present invention is administered through a gastric fistula in a state where the composition is dissolved in water at 0.5 kcal/mL to 1.5 kcal/mL and preferably 1 kcal/mL. The dose is usually about 500 kcal to 3000 kcal per person per day in terms of the amount of energy. The dose can be appropriately increased and decreased depending on the clinical condition, the age, the weight, or the like of a gastrostomy-tube patient. The dose of the nutritional composition according to the present invention per person per day is preferably greater than or equal to 900 kcal, more preferably 900 kcal to 3000 kcal, and still more preferably 900 kcal to 2500 kcal. The nutritional composition for gastrostomy-tube patients according to the present invention may be administered to infants or children, but preferably is administered to adults, and more preferably is administered to elderly people of 60 years of age or older. When the composition is administered to elderly people, the nutritional composition according to the present invention is preferably administered about at least 900 kcal per day.

The injection rate of the nutritional liquid composition according to the present invention is adjusted depending on the clinical condition of a gastrostomy-tube patient. In general, the injection rate is 75 mL/h to 100 mL/h. In addition, the composition is preferably injected into the gastric fistula in a posture in which the bed is further raised by about 30 degrees above the dorsal position in order to further suppress vomiting, and the posture is preferably maintained over a certain time (for example, about one hour) after the injection.

The nutritional composition according to the present invention is in a liquid form when being administered, and therefore, it is preferable that the composition be prepared in a dosage form which can be dissolved in a suitable amount of water or the like when in use. Examples of the dosage form which can be dissolved in a suitable amount of water or the like include powdered medicines, fine granules, granules, tablets, and capsules. These formulations can be prepared through usual methods by using active ingredients as they are; mixing and granulating the active ingredients with pharmaceutically and pharmacologically acceptable additives depending on each dosage form; emulsifying or suspending the active ingredients by dissolving the active ingredients in an appropriate solvent; and mixing the active ingredients with an appropriate base. The solvent is mainly purified water, but it is also possible to use ethanol, glycerol, propylene glycol, and the like.

Examples of the additives which can be added to powdered medicines, fine granules, granules, tablets, and capsules include excipients (for example, lactose, glucose, D-mannitol, starch, crystalline cellulose, calcium carbonate, kaolin, light anhydrous silicic acid, and trehalose), binders (for example, starch paste solutions, gelatin solutions, hydroxypropyl cellulose, hydroxypropyl cellulose, polyvinyl pyrrolidone, and ethanol), disintegrating agents (for example, starch, gelatin powder, carboxymethyl cellulose, and carboxymethyl cellulose calcium salt), lubricants (for example, magnesium stearate and talc), and coating agents (for example, hydroxypropyl cellulose, hydroxypropyl methylcellulose, acetyl cellulose, white sugar, and titanium oxide), and colorants, flavoring or odor-adjusting agents and the like can be added thereto as necessary. In addition, examples of the additives which can be added to liquid medicines for internal use include preservatives (for example, benzoic acid, p-hydroxybenzoate, and sodium dehydroacetate), suspending or emulsifying agents (for example, gum arabic, tragacanth, sodium carboxymethylcellulose salt, methylcellulose, egg yolk, and surfactants), and sweeteners/acidulating agents (for example, trehalose and citric acid), and colorants, stabilizers, and the like can be added as necessary.

In addition, in a case where the nutritional composition according to the present invention is in a dosage form which can be dissolved in a suitable amount of water or the like, the composition may be prepared in a liquid form by being dissolved in a suitable amount of water or the like immediately before being administered to a patient; or administered to a patient after preparing the composition in a liquid form in advance and storing the prepared composition at low temperature (refrigerated or frozen depending on the storage period).

The nutritional composition according to the present invention is preferably prepared as a liquid agent. For example, each component may be dissolved or suspended in water or the like such that the amount of each component becomes an adequate amount and the obtained liquid agent may be sealed in an airtight container, in an aseptically managed preparation facility the day before the administration or the like, and then delivered to a hospital or a house of a patient to whom the prepared composition is to be administered while storing the prepared composition at low temperature (refrigerated or frozen depending on the storage period).

The nutritional composition according to the present invention may be prepared once in an amount for a plurality of people or multiple administrations. For example, a large amount of the nutritional composition can be prepared once by adding a liquid such as water to a container filled with a solid composition from which it is possible to prepare the nutritional composition in an amount that can be administered plural times by adding the liquid, to be administered to a plurality of patients in the facility by being dispensed to a container for administration through a gastric fistula.

### <Composition for Nutritional Composition for Gastrostomy-Tube Patients Which Can be Administered Through Tube>

A composition for a nutritional composition for gastrostomy-tube patients which can be administered through a tube according to the present invention (hereinafter, in some cases, referred to as a “composition according to the present invention”) is a composition which is used for preparing the nutritional liquid composition for gastrostomy-tube patients which can be administered through a tube by being mixed with water or other components as necessary when in use; and is stored in a multi-chamber container. At least a part of the composition according to the present invention is in a solid form, and therefore, the composition according to the present invention has an excellent storage stability.

In general, in nutrition supply through a gastric fistula, an excessively high concentration of the nutritional composition easily induces pulmonary aspiration, and therefore, the nutritional composition is prepared at a concentration of 0.5 kcal/mL to 1.5 kcal/mL. As a result, a certain amount of nutritional liquid composition is required in order meet the required amount of nutrition per day. The composition according to the present invention is delivered to a medical facility, a nursing facility, or the like in a solid form or the like in a state of having a relatively smaller volume, and then, a large amount of water is initially supplied when in use to prepare the nutritional composition. Therefore, the composition is beneficial in that handling during transportation or the like is simple as well as long-term stable preservation being possible.

The composition according to the present invention is a composition for preparing the nutritional composition for gastrostomy-tube patients which contains a protein source, a carbohydrate source, and a lipid source. That is, the composition according to the present invention contains all or a part of a raw material of a target nutritional composition for gastrostomy-tube patients which can be administered through a tube except for water. As the protein source, a protein such as casein may be used or only amino acids may be used. The target nutritional composition for gastrostomy-tube patients which can be administered through a tube and is prepared from the composition according to the present invention preferably contains only amino acids as the protein source. The nutritional composition according to the present invention which is prepared in a liquid so as to be able to be administered through a gastric fistula is more preferable.

The composition according to the present invention is a composition that can be used to prepare a target nutritional liquid composition in a multi-chamber container in which the composition is stored. Furthermore, the prepared nutritional composition can be administered to a gastric fistula through a tube or the like as it is. In a case where a composition for preparation, which is required for preparing a nutritional composition for gastrostomy-tube patients for one patient, that is, a single dose or a dose for one day, is stored in one multi-chamber container, the multi-chamber container after the preparation is used as a container for administration through a gastrostomy-tube as it is. In addition, the composition according to the present invention may be stored in one multi-chamber container in an amount from which a dose for a plurality of people is capable of being prepared or a dose for a multiple administrations in one multi-chamber container.

Specifically, the composition according to the present invention is stored in a multi-chamber container which has at least two chambers which are partitioned by a communicating partition wall portion formed of a flexible film. The multi-chamber container is provided with one water injection chamber and one or a plurality of medicine storage chambers.

In a case where the multi-chamber container includes one medicine storage chamber, the composition according to the present invention is stored in the one medicine storage chamber in a state where the whole amount of the composition is in a solid form. In a case where there are two or more medicine storage chambers, the composition according to the present invention may be stored by being divided between the medicine storage chambers. However, at least one medicine storage chamber can store the composition in a solid form. All of the medicine storage chambers may store the composition in a solid form. One medicine storage chamber may store the composition in a solid form and other medicine storage chambers may store a relatively small amount of the composition in a liquid form, for example, 1 mL to 250 mL.

The water injection chamber in the multi-chamber container is a cavity, which has a capacity of storing greater than or equal to 200 mL of a liquid, and has an attached water injection port, which has a resealable mechanism that can inject and discharge a liquid. The capacity of the water injection chamber can be appropriately adjusted in accordance with the amount of a target nutritional composition for gastrostomy-tube patients that is to be finally prepared. For example, in a case of preparing a single dose when the composition is taken three times per day, the capacity of the chamber can be set such that 200 mL to 500 mL of a liquid can be stored therein. In a case of preparing a dose for one day, the capacity of the chamber can be set such that 800 mL to 2000 mL of a liquid can be stored therein. In a case of preparing an amount of the composition for multiple administrations or a plurality of people, the capacity of the chamber can be set such that a larger amount of a liquid can be stored therein. The volume of a liquid which can be stored in the water injection chamber is preferably greater than or equal to 300 mL, more preferably greater than or equal to 800 mL, still more preferably greater than or equal to 1000 mL, and still more preferably 1000 mL to 3000 mL.

The water injection chamber provided in the multi-chamber container is a cavity in which a solid or a liquid is not stored. In the present invention, there is preferably substitution with an inert gas in the water injection for the purpose of storage stability of the composition according to the present invention, which is stored in the medicine storage chamber. Examples of the inert gas particularly include a nitrogen gas or a helium gas, and a nitrogen gas is preferable.

In order to prepare a nutritional liquid composition for gastrostomy-tube patients which can be administered through a tube using the composition according to the present invention, first, the water injection port is open and an appropriate amount of water, which is required for preparing the desired nutritional composition, is injected therethrough, and then, the water injection port is resealed. A partition wall portion allows communication between the water injection chamber and at least one medicine storage chamber in the multi-chamber container in the state where the water injection port is sealed, and the water is mixed with the composition according to the present invention, which has been stored in the medicine storage chamber. Accordingly, it is possible to prepare a nutritional composition for gastrostomy-tube patients which can be administered through a tube in the multi-chamber container which is blocked and sealed from the outside. Therefore, it is possible to markedly reduce a risk of bacterial infection due to falling bacteria or the like during the preparation. At this time, it is possible to prepare a nutritional composition for gastrostomy-tube patients which contains all components which have been stored in the multi-chamber container, by the whole partition wall portion allowing communication in the multi-chamber container. In addition, in a case where the multi-chamber container has two or more medicine storage chambers, the nutritional composition for gastrostomy-tube patients may be prepared without containing a component which has been stored in a part of the medicine storage chambers. In this case, a partition wall portion between the medicine storage chamber, which stores the component that is not to be present in a final nutritional composition, and other chambers, may not allow communication, but the remaining partition wall portions may allow communication.

The partition wall portion in the multi-chamber container divides the chambers and can allow communication therebetween when preparing the nutritional composition for gastrostomy-tube patients after water is injected into the water injection chamber. The partition wall portion can be formed, for example, by releasably welding (weakly sealing) inner surfaces of two flexible films constituting the multi-chamber container. In addition, it is also possible to form the partition wall portion by providing a breakable portion in a flexible film separately from a main body of the multi-chamber container.

It is preferable that the partition wall portion allows communication when pressing the water injection chamber from the outside in a state where the water injection port is sealed, after water is injected into the water injection chamber. In a case of an easily peeled seal of which the partition wall portion is formed through weak sealing, the partition wall portion is preferably formed with a welding force to the extent that two flexible films which constitute the main body of the multi-chamber container are separated from each other by pressing the water injection chamber, into which water is injected, from the outside. Moreover, the partition wall portion is more preferably formed with a welding force to the extent that the partition wall portion first allows communication after pressing the water injection chamber, into which water is injected, from the outside without the partition wall portion allowing communication by simply injecting water into the water injection chamber. It is possible to divide the time point into a time point at which water is injected into the water injection chamber and a time point at which the nutritional liquid composition for gastrostomy-tube patients is prepared, by the partition wall portion first allowing communication due to the water injection chamber being pressed from the outside without allowing the partition wall portion to be communicate by simply injecting water into the water injection chamber. For example, in a preparation chamber in a medical facility or a nursing facility (or may also be in a separate facility positioned near these facilities), water is injected into the water injection chamber in the multi-chamber container and other components are injected into the multi-chamber container as necessary; the multi-chamber container is carried to a bedside of a patient in a state where the partition wall of the multi-chamber container, in which all raw materials are stored, is maintained; and then, the partition wall is made to allow communication due to the water injection chamber being pressed from the outside to prepare the nutritional liquid composition for gastrostomy-tube patients.

The water injection port which is provided in the water injection chamber is a member capable of discharging a liquid and includes a resealable mechanism. Examples of such a port include a port configured to have a pipe member (tubular member) which has a rigidity that can maintain its form and a lid member capable of opening and closing an opening of the pipe member. A screw portion is preferably formed on the outer surface of the pipe member and the inner wall surface (surface coming into contact with the outer wall surface of the pipe member) of the lid member, so as to thread and mount both the surfaces together.

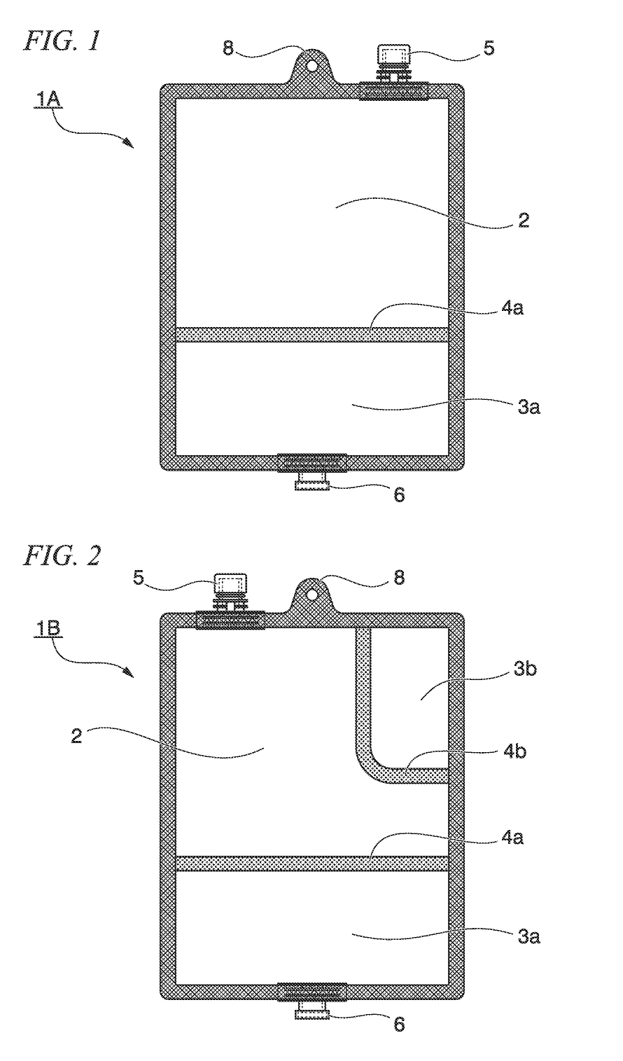

FIG. 1 is a side view of a multi-chamber container 1A as an example of a multi-chamber container (hereinafter, in some cases, referred to as “multi-chamber container according to the present invention”) for storing a composition according to the present invention. The main body (bag body) of the multi-chamber container 1A is formed such that two planar flexible films in almost identical shapes are fluid-tightly and firmly (unpeelably) welded by overlapping each other and heat-sealing the outer circumferential portions thereof; and a partition wall portion 4a is formed by weakly sealing the facing surfaces of the flexible films in order to divide the inside into a plurality of chambers (water injection chamber 2 and medicine storage chamber 3a). Instead of the two overlapping planar flexible films, one planar flexible film may be formed by being folded and overlapping, or a cylindrical flexible film may be used. As the flexible film constituting the main body of the multi-chamber container, for example, a resin film such as polyethylene and polypropylene can be used. The flexible film may be a monolayer film formed of one kind of a resin, or may be a multilayer film.

A water injection port 5 which is configured to have a pipe member and a lid member that can be threaded to each other is provided in the outer circumferential portion of the main body of the multi-chamber container so as to be open to the water injection chamber 2. The water injection port 5 is constituted by interposing and welding the pipe member at the same time as when welding the outer circumferences of the two flexible films constituting the main body of the multi-chamber container, therebetween. As the water injection port 5, a water injection port which is easily welded to the flexible films constituting the main body of the multi-chamber container is preferable and a water injection port which is made of the same material as those of the flexible films is more preferable.

In a multi-chamber container 1A, a discharge port 6, which allows communication with a tube that is connectable to a gastric fistula, is provided in the outer circumferential portion of the main body of the multi-chamber container. The discharge port 6 is constituted by being interposed and welded at the same time as when welding the outer circumferences of the two flexible films constituting the main body of the multi-chamber container, therebetween. “Allowing communication with a tube that is connectable to a gastric fistula” includes allowing communication with a tube that is directly connected to a gastric fistula through an appropriate connector, in addition to allowing communication with a tube that is indirectly connected to a gastric fistula.

Regarding the discharge port 6, the present invention is not particularly limited as long as the inside of the multi-chamber container can be liquid-tightly blocked from the outside and the discharge port allows communication with a tube that is connectable to a gastric fistula, after a nutritional composition for gastrostomy-tube patients which can be administered through a tube is prepared in the multi-chamber container, until the time when the nutritional composition for gastrostomy-tube patients is administered to a patient, and any discharge port can be used which is usually used in a container that stores an infusion solution, a tube feeding nutritional solution, or the like. As the discharge port, a hollow tubular member which can be inserted into a tube and a coating member (for example, a rubber cap or the like) with which the hollow tubular member is liquid-tightly coated may be used; a hollow tubular member which can be inserted into a tube and a resealable lid member which covers an end portion of the hollow tubular member may be used; or a discharge port may be used such that an outer circumferential portion of a main body of a container is formed in a shape which allows communication with a tube which is connected to a cut cross section of a fixed portion of the outer circumferential portion. In addition, a pipe member which includes a cylindrical space and a rubber plug that blocks the cylindrical space can be used as the discharge port. In this case, the tube and the multi-chamber container can be made to communicate with each other by forming a hollow needle-(injection needle-) like member at a tip end of a tube which is connected to a gastric fistula and by piercing the rubber plug in the discharge port with the hollow needle-like member.

In the multi-chamber container 1A, the discharge port 6 is provided so as to be open to the medicine storage chamber 3a. However, the discharge port may be open to any chamber in the multi-chamber container since all of the partition wall portions may allow communication in the multi-chamber container when discharging a liquid composition in the multi-chamber container to a gastric fistula. For example, in the multi-chamber container 1A, the discharge port 6 may be provided so as to be open to the water injection chamber 2. In addition, in a case where the water injection port 5 allows communication with a tube that is connectable to a gastric fistula, a liquid may be discharged from the water injection port 5 to the gastric fistula, and therefore, it is unnecessary to provide the discharge port 6.

A hanging hole 8 may be further provided in the multi-chamber container 1A. The hanging hole 8 is a through hole which is provided in a fixing portion in the outer circumferential portions of flexible films constituting the main body of the multi-chamber container. The hanging hole 8 may be provided at a place such as the outer circumferential portion of the main body of the multi-chamber container. However, the hanging hole is preferably provided on a side opposite to the portion provided with the discharge port 6 such that the discharge port 6 is positioned in a lower portion of the multi-chamber container when the multi-chamber container is hung by the hanging hole 8 using a hanging tool.

There is only a medicine storage chamber 3a in the multi-chamber container 1A, as a medicine storage chamber. Therefore, the total amount of the composition according to the present invention is stored in the medicine storage chamber 3a in a solid form. The solid form may be in any of a powdered form, a granular form, a tablet form, and the like, or a plurality of forms may be mixed.

In the case of the composition which is stored in the multi-chamber container 1A, the solid composition which is stored in the medicine storage chamber 3a and a solution which is injected through the water injection port 5 are mixed to prepare a nutritional liquid composition for gastrostomy-tube patients.

In a case where all of components, except for water, for preparing a target nutritional liquid composition for gastrostomy-tube patients are stored in the medicine storage chamber 3a, it is possible to prepare the target nutritional liquid composition by injecting only water through the water injection port 5. In this case, as the solid composition which is stored in the medicine storage chamber 3a, a nutritional composition according to the present invention which is prepared in a solid form is preferable and a solid composition which has the composition shown in Table 1 is more preferable.

Sterilized water is preferable as water to be injected into the water injection chamber 2 from the water injection port 5, but water may be used which has not been subjected to a sterilization treatment. In addition, the temperature of water to be injected may be at room temperature (1° C. to 30° C.) or may be tepid (30° C. to 40° C.).

Components other than water may be injected into the water injection chamber 2 together with water or separately from water. For example, water and mineral components can be injected into the water injection chamber 2. In this case, a saline solution or an electrolyte infusion agent may be injected, or a small amount of an aqueous solution or a tablet which contains a mineral component may be put into the water injection chamber 2 through the water injection port 5 separately from water. As the mineral component, for example, a sodium agent, a potassium agent, a calcium agent, an alkaline agent, and the like which are used in infusion or the like can be appropriately used. In addition, a trace element such as an iron preparation or the like may be injected thereinto.

In addition, components, such as antiflatulent or a medicine for treating a patient, which need to be administered to each patient in an adequate amount may be stored in the medicine storage chamber 3a in a solid form together with other compositions in advance, but are preferably injected into the water injection chamber 2 from the water injection port 5. Among components which are contained in a target nutritional liquid composition for gastrostomy-tube patients, components that will be administered to many patients in substantially the same doses can be stored in the multi-chamber container in advance, and components which will be administered in different doses, or the like for each patient are injected through the water injection port 5 at the time of preparation for use. Therefore, it is possible to simply prepare a custom-made nutritional composition for gastrostomy-tube patients.

In addition, components such as vitamins which have low storage stability may be stored in the medicine storage chamber 3a in advance, but are preferably injected into the multi-chamber container through the water injection port 5 or the like at the time of preparation for use instead of being stored in the medicine storage chamber 3a. For example, a commercially available multivitamin preparation or the like may be injected into the water injection chamber 2, or a solution which contains only one type of or a plurality of types of vitamins may be injected thereinto.

Only a part of components, other than water, for preparing a target nutritional liquid composition may be stored in the medicine storage chamber 3a, and the remaining components may be injected into the water injection chamber 2 through the water injection port 5 together with water or separately from water. The composition according to the present invention which is stored in the medicine storage chamber 3a preferably contains at least a carbohydrate source and more preferably contains dextrin, in particular, maltodextrin.

In a case where the target nutritional composition for gastrostomy-tube patients contains amino acids as a protein source, it is preferable that the composition according to the present invention which is stored in the medicine storage chamber 3a include at least L-glutamine among the amino acids. L-glutamine readily decomposes in an aqueous solution, but is stable in a solid form. Therefore, L-glutamine can be stably stored over a long period of time by being stored in the medicine storage chamber 3a as a solid composition. The solid composition which is stored in the medicine storage chamber 3a preferably contains one kind or two or more kinds of branched-chain amino acids (L-valine, L-isoleucine, or L-leucine) in addition to L-glutamine.

In a case where the solid composition which is stored in the medicine storage chamber 3a does not contain amino acids other than L-glutamine, amino acids which are required at the time of preparation for use may be injected into the water injection chamber 2 from the water injection port 5. Particularly, amino acids which readily react with other compounds such as glutamic acids or cysteine may be stored in the medicine storage chamber 3a in advance, but are preferably injected into the multi-chamber container through the water injection port 5 or the like at the time of preparation in use without being included in the medicine storage chamber 3a. For example, it is possible to inject a commercially available amino acid agent into the water injection chamber 2. Examples of the commercially available amino acid agent include AMINIC (manufactured by Ajinomoto Pharmaceuticals Co., Ltd.) or AMIPAREN (manufactured by Otsuka Pharmaceutical Factory, Inc.), which are liquid agents that contain amino acids other than L-glutamine.

Water or other components are preferably injected into the water injection chamber 2 from the water injection port 5 such that various bacteria or the like are not mixed in the water injection chamber 2. For example, water or other components can be injected into the water injection chamber 2 in a clean room which is provided in a medical facility or the like. In a case where there is no clean room, various bacteria can be prevented from being mixed in, for example, by wiping the surface of the water injection port 5 or the like using ethanol for disinfection before opening and closing the water injection port 5.

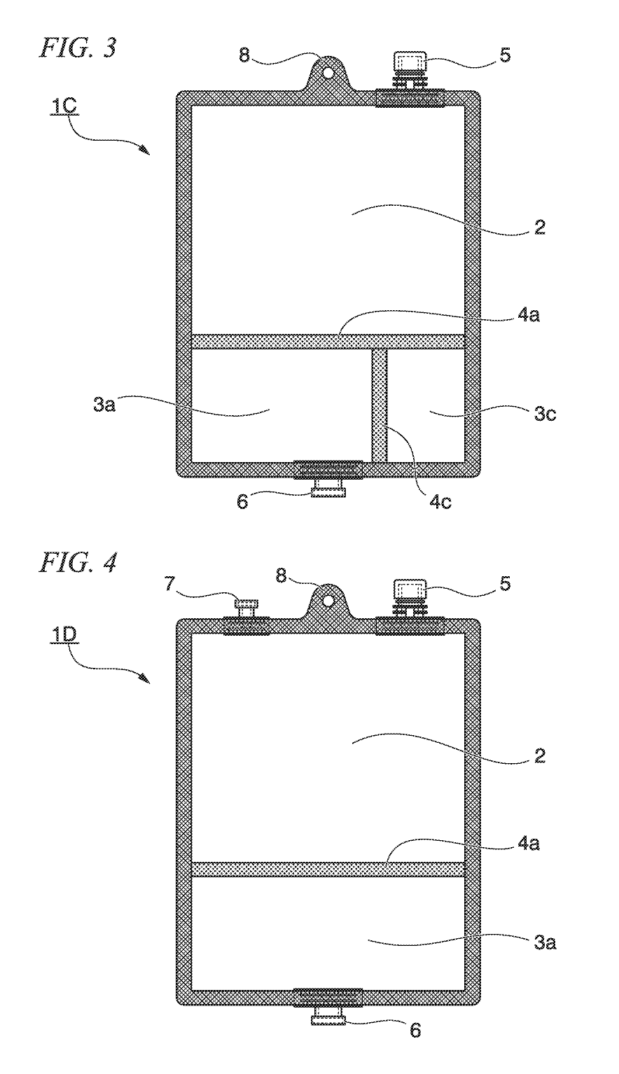

The multi-chamber container according to the present invention may have two or more medicine storage chambers. In a case where the multi-chamber container includes a plurality of medicine storage chambers, each medicine storage chamber may be adjacent only to the water injection chamber 2 like medicine storage chambers 3a and 3b in the multi-chamber container 1B shown in FIG. 2, or may be adjacent to both of the water injection chamber 2 and the other medicine storage chamber like medicine storage chambers 3a and 3c in multi-chamber container 1C shown in FIG. 3.

It is possible to separately store components which readily decompose in a state of being mixed with other components, by providing two or more medicine storage chambers. By doing this, it is possible to improve long term stability of the composition according to the present invention.

In addition, in a case where there are two or more medicine storage chambers, at least a composition which is stored in one medicine storage chamber may be in a solid form, and a component which is stored in the remaining medicine storage chamber may be in a solid form or a liquid form. For example, in the multi-chamber container 1B, it is possible to store a solid composition, which contains dextrin and L-glutamine, in the medicine storage chamber 3a and to store the remaining solid composition, which contains a protein source and a lipid source, in the medicine storage chamber 3b. In addition, it is possible to include amino acids, which contain dextrin and L-glutamine, in medicine storage chamber 3a and to store a milk-like composition (for example, a fat content of 10 mass/volume % to 20 mass/volume %) in which a lipid source is emulsified, in the medicine storage chamber 3b. As the milk-like composition, for example, it is possible to use a commercially available fat emulsion.

It is preferable that a nutritional composition with a comparatively lower fat content be administered to a gastrostomy-tube patient who is at a high risk of diarrhea or aspiration pneumonia, but it is preferable that a nutritional composition with a higher fat content be administered to a gastrostomy-tube patient who is at a low risk of diarrhea or aspiration pneumonia due to improved symptom or the like, in terms of supply of sufficient nutrition. For this reason, in the multi-chamber container for storing compositions being a three-chamber container such as the multi-chamber container 1B and multi-chamber container 1C, it is preferable that as the composition according to the present invention, a solid composition, which contains a protein source, a carbohydrate source, and a lipid source at a comparatively low concentration, be stored in one medicine storage chamber and a fat source be stored in the other medicine storage chamber. Moreover, it is more preferable that a solid composition, which contains only amino acids as a protein source, a carbohydrate source, and a lipid source at a comparatively low concentration, be stored in one medicine storage chamber and a fat composition such as a fat emulsion be stored in the other medicine storage chamber. Moreover, it is still more preferable that a nutritional composition according to the present invention in a solid form be stored in one medicine storage chamber and a fat source be stored in the other medicine storage chamber. Moreover, it is particularly preferable that ELENTAL (manufactured by Ajinomoto Pharmaceuticals Co., Ltd.) in a powdered form be stored in one medicine storage chamber and a fat composition such as a fat emulsion be stored in the other medicine storage chamber. It is possible to use the compositions separately when preparing a nutritional composition to which fat is added and when preparing a nutritional composition to which fat is not added, in accordance with the condition of patients by storing a composition, which is required for preparing a nutritional composition favorable for a gastrostomy-tube patient at a high risk of diarrhea or aspiration pneumonia, in a first medicine storage chamber and storing a fat composition such as a fat emulsion in a second medicine storage chamber.

In a case where there is a possibility that a target nutritional liquid composition will be prepared without using components of the composition according to the present invention which are stored in some of a plurality of medicine storage chamber depending on the condition of a target patient, the discharge port or an injection port, which is to be described later, is provided so as to be open to the water injection chamber, or is provided so as to be open to the water injection chamber with which the partition wall portion between the water injection chamber and the medicine storage chamber necessarily communicates at the time of preparing the target nutritional liquid composition.

In addition, in a case where there are two or more medicine storage chambers, it is possible to separately store a component for nutrition supply and a component, to be mixed with the component for nutrition supply, for a purpose other than for the nutrition supply. For example, in the multi-chamber container 1B, it is possible to store a solid composition, which contains a protein source, such as a protein source which consists of only amino acids, a carbohydrate source, and a lipid source, in the medicine storage chamber 3a, and to store an antiflatulent, or medicines, such as an antibiotic preparation, an antipyretic analgesic anti-inflammatory agent, and an antitumor agent, for treating diseases, in the medicine storage chamber 3b. One kind or two or more kinds of the components, to be mixed with the component for nutrition supply, for a purpose other than the nutrition supply which is stored in the medicine storage chamber 3b may be mixed with the component for nutrition supply.

In the multi-chamber container according to the present invention, the water injection port is provided for injecting a comparatively larger amount, for example, greater than or equal to 200 mL, of water in order to prepare a nutritional liquid composition for gastrostomy-tube patients. An injection port 7 may be provided separately from the water injection port 5 as shown in multi-chamber container 1D in FIG. 4 in order to inject a smaller amount of a component into the multi-chamber container. Injection of various components through the injection port may be performed before injecting water into the water injection chamber 2; after injecting water and before the partition wall portion 4a allows communication; or after the partition wall portion 4a allows communication.

As the injection port 7, for example, a water injection port is preferable, through which it is possible to inject a liquid composition or emulsified composition in a volume of less than or equal to 100 mL which has been subjected to a sterilization treatment while maintaining the sterilized state. Examples of the injection port 7 include a pipe member which is provided with a cylindrical space and a rubber plug that blocks the cylindrical space. In this case, it is possible to fill a syringe, to which a hollow needle is attached, for example, injection needle or communication needle, at a tip end, with a liquid composition or an emulsified composition which have been sterilized and to inject the composition, with which the syringe is filled, into the multi-chamber container; and to reseal the rubber plug after the injection by withdrawing the hollow needle from the rubber plug. In multi-chamber container 1D in FIG. 4, the injection port 7 is provided such that the cylindrical space thereof opens to the water injection chamber 2, but may open in the medicine storage chamber 3a.

It is preferable that the multi-chamber container according to the present invention be subjected to a sterilization treatment in advance before the composition according to the present invention is stored therein. As the sterilization treatment, it is possible to appropriately select and perform a method from well-known methods as a sterilization method, such as radiation sterilization treatment, using a plastic bag or the like.

The composition according to the present invention which is stored in the multi-chamber container is preferably subjected to a sterilization treatment for the purpose of long-term storage stability, reduction of bacterial infection, or the like. For example, after the multi-chamber container according to the present invention is subjected to a sterilization treatment in advance, the composition according to the present invention which has been subjected to a sterilization treatment similarly to the multi-chamber container is aseptically stored and sealed. In addition, the multi-chamber container may be subjected to the sterilization treatment after sealing the composition according to the present invention by storing the composition according to the present invention in the multi-chamber container. As the sterilization treatment, it is possible to appropriately select and perform a method from a well-known sterilization methods, such as radiation sterilization treatment, using a liquid medicine container, such as a plastic bag, which is filled with a liquid medicine.

The multi-chamber container in which the composition according to the present invention is liquid-tightly stored may be distributed on the market as it is, or in a state of being air-tightly stored in an external packaging bag together with a deoxygenating agent and/or a drying agent. Decomposition, deformation, discoloration, or the like of the composition stored in the multi-chamber container is suppressed by storing the multi-chamber container under a low oxygen environment, and therefore, it is possible to further improve the long-term storage stability. As the deoxygenating agent or the drying agent, for example, it is possible to use a commercially available deoxygenating agent or drying agent.

The external packaging bag is preferably formed of a flexible film which has gas-impermeable properties and is more preferably formed of a flexible film which has light-shielding properties and gas-impermeable properties. Examples of the flexible film which has light-shielding properties and gas-impermeable properties include a multilayer film which is obtained by stacking a light-shielding layer, such as a metal deposition layer, a metal foil layer, or a silica deposition layer, on the flexible film which has gas-impermeable properties. Examples of the flexible film which has gas-impermeable properties include a monolayer or multilayer film which includes a layer formed of polyesters such as polyvinylidene chloride or polyethylene telephthalate.

In facilities such as medical facilities or nursing facilities where there are patients, it is possible to prepare a nutritional composition for gastrostomy-tube patients which can be administered through a tube, by injecting water and other components as necessary into a multi-chamber container which store the composition according to the present invention through a water injection port or an injection port and by mixing all of required components in a state where the multi-chamber container is sealed. The prepared nutritional liquid composition is administered to a gastric fistula by the discharge port (or water injection port) allowing communication with a tube which is connected to the gastric fistula, from the multi-chamber container. At the time of administering the composition to the gastric fistula, the multi-chamber container opens only to the tube which is connected to the gastric fistula. Therefore, the risk of developing an infectious disease due to various bacteria such as falling bacteria is significantly reduced. In this manner, it is possible to perform preparation of a nutritional composition or administration of the prepared nutritional composition to a gastric fistula in a state where the risk of various bacteria being mixed in is reduced, and therefore, the composition according to the present invention is favorable also for nutrition supply through a gastric fistula in home care as well as in medical facilities or nursing facilities.

### EXAMPLES

Next, the present invention will be described in more detail with reference to Examples or the like, but the present invention is not limited thereto.

### <Statistical Treatment>

In the following Examples or the like, a measured value is represented by an average value±SD. A statistical treatment in which measurement results of two groups are compared with each other was performed as follows. First, Yates' chi-square test for performing correction for continuity was performed as necessary in order to compare category data with each other. In a case where the number of cases was small, Fisher's exact test was used. In a case where average values of the two groups were compared with each other for parametric data, a Student's t-test was used.

### Example 1

A compositional nutrient which consisted of amino acids as a protein source and a semi-digested nutrient of which the protein source was proteins or peptides were respectively administered to bedridden gastrostomy-tube patients, that is, patients who necessarily sleep in a bed all day, in hospital through gastric fistulas for 20 months, and the development frequency of aspiration pneumonia or the frequency of nutrient absorption treatment was checked. As the compositional nutrient, 1 kcal/mL (760 mOsm/L) of ELENTAL (manufactured by Ajinomoto Pharmaceuticals Co., Ltd.) (content of amino acids: 176 g/kg, content of dextrin (average molecular weight: 900): 794 g/kg, content of soybean oil: 6 g/kg, content of vitamins and minerals: 24 g/kg) which was composed of the composition shown in Table 1 and was prepared by being dissolved in water was used. As the semi-digested nutrient, ENSURE LIQUIDO (manufactured by Abbott Japan Co., Ltd.) (which was 1 kcal/mL suspension and of which the content of protein was 18 W/W %, the content of carbohydrate was 62 W/W %, the content of lipid was 20 W/W %, per dry weight) which mainly included casein as a protein source and had been widely used as a nutritional composition for gastrostomy-tube patients, was used.

### <Gastrostomy-Tube Patients>

Tests were performed on bedridden gastrostomy-tube patients who were hospitalized due to development of aspiration pneumonia, urinary tract infection, or bile duct infection. Clinical features of a compositional nutrient administration group and a semi-digested nutrient administration group are shown in Table 2. In Table 2, “CVA” represents cerebral apoplexy, “CNSD” represents a central nervous system disease, and “p value” represents a probability that the null hypothesis, that there may be no difference between both the administration groups in each item, is satisfied. There was no obvious difference between both the administration groups in terms of the baseline of clinical parameters such as the age, the gender, the reason for hospitalization, application to PEG, or the like.

TABLE 2

| | Compositional | Semi-digested | |
|---|---|---|---|
| | nutrient | nutrient | |
| | administration | administration | |
| | group | group | p value |
| Number of cases | 60 | 67 | |
| Number of male patients | 33 | 27 | 0.098 |
| Average age (years) | 79 ± 8 | 81 ± 7 | 0.88 |
| Minimum age (years) | 61 | 62 | |
| Maximum age (years) | 92 | 94 | |
| Application to PEG | 48/12 | 57/10 | 0.45 |
| (CVA/CNSD) | | | |

Reason for hospitalization

| | | | |
|---|---|---|---|
| pneumonia | 43 | 47 | 0.95 |
| Bile duct infection | 12 | 15 | |
| Urinary tract infection | 5 | 5 | |

### <Administration Method>

A compositional nutrient or a semi-digested nutrient was administered to each patient through a gastric fistula at an identical rate (3.3 mL/min to 5.0 mL/min) by natural dripping due to gravity. All of the nutrients were adjusted so as to be administered for 60 minutes to 90 minutes at all times. The frequency, at which nutrient absorption treatment through the trachea was performed, the development frequency of aspiration pneumonia, and the number of times of defecation were recorded during the administration period. After checking for the presence and absence of the development of aspiration pneumonia by performing a chest X-ray on patients who had symptoms in the respiratory system, nutrition supply from the gastric fistula was stopped and adequate treatment was performed as necessary. In a case where the number of times of defecation was greater than or equal to five per day continuously for one week even though there was no sign of acute intestinal infection, it was assumed that diarrhea had developed, which was induced by the nutrition supply through a gastric fistula. All of the signs starting with aspiration pneumonia were measured only once per patient.

### <Results>

In each administration group, the nutritional requirements (kcal) per day, the nutrition period (months), the number of patients who were subjected to a nutrient absorption treatment through the trachea was performed, the number of patients who developed aspiration pneumonia, and the number of patients who developed diarrhea due to nutrition supply through a gastric fistula are shown in Table 3. The results show that in the compositional nutrient administration group, pulmonary aspiration, aspiration pneumonia, and the incidence rate of diarrhea due to nutrition supply through a gastric fistula were more significantly decreased than those in the semi-digested nutrient administration group. Whereas there were no patients who developed pulmonary aspiration, aspiration pneumonia, and diarrhea due to nutrition supply through a gastric fistula through continuous administration over an average of 21 months in the compositional nutrient administration group, nutrient absorption treatments were performed on 8 patients in the semi-digested nutrient administration group, and among them, 5 patients developed aspiration pneumonia (incidence rate: 7.5%) and 4 patients out of 5 patients who developed aspiration pneumonia developed diarrhea. There were no patients who developed only diarrhea without development of aspiration pneumonia. In addition, 4 patients out of 8 patients who were subjected to a nutrient absorption treatment, 4 patients out of 5 patients who developed aspiration pneumonia, and 3 patients out of 4 patients who developed diarrhea had a medical history of aspiration pneumonia.

TABLE 3

| | Compositional | | Semi-digested | | |
|---|---|---|---|---|---|
| | nutrient | | nutrient | | |
| | administration | | administration | | |
| | group | | group | | p value |
| Average nutritional | 900 ± 0 | kcal | 1040 | kcal | 0.76 |
| requirements per day | | | | | |
| Nutrition period | 21 ± 4 | months | 17 ± 4 | months | 0.66 |
| Nutrient absorption | 0 | patients | 8 | patients | 0.0057 |
| treatment | | | | | |
| Aspiration pneumonia | 0 | patients | 5 | patients | 0.031 |
| Diarrhea | 0 | patients | 4 | patients | 0.054 |

That is, in the present Example, it was found that aspiration pneumonia rarely developed due to a nutritional composition which contains only amino acids as a protein source and of which the lipid content was low, and even in a case where the nutritional composition was continuously administered over 20 months or longer through a gastric fistula, the incidence rate of aspiration pneumonia and diarrhea was suppressed to 3% or less and preferably 1% or less. It is obvious from the results that the nutritional composition for gastrostomy-tube patients according to the present invention can suppress the development of aspiration pneumonia and can be continuously administered over a long period of time.

In addition, even though there was no obvious difference between both administration groups as shown in Table 2, there were 8 patients who required the nutrient absorption treatment in the semi-digested nutrient administration group whereas there were no patients who required the nutrient absorption treatment in the compositional nutrient administration group. It was inferred that this was because the discharge rate of the compositional nutrient from the stomach was higher than that of the semi-digested nutrient as shown in Example 2 to be described later. It is obvious from the result that administration of the nutritional composition for gastrostomy-tube patients according to the present invention which contains only amino acids as a protein source can more significantly suppress the development of aspiration pneumonia and is more excellent in safety than administration of the semi-digested nutrient, with respect to gastrostomy-tube patients with decreased cough reflex, and among them, gastrostomy-tube patients who have decreased cough reflex and are easily infected with infection.

### Example 2

The discharge rate of the compositional nutrient and the semi-digested nutrient using Example 1 from the stomach was determined.

### <Gastrostomy-Tube Patients>

Tests were performed on 19 bedridden gastrostomy-tube patients who were hospitalized and had a medical history of development of aspiration pneumonia. The criteria for exclusion included patients who taken benzodiazepine or opioids regularly; patients who had clinical evidence of acute infection; patients who have had abdominal surgery; and patients in Class 4 or 5 of Physical Status Classification of American Society of Anesthesiologists. Clinical features of 19 gastrostomy-tube patients are shown in Table 4.

TABLE 4

| | Gastrostomy-tube |
|---|---|
| | patients |
| Number of cases | 19 |
| Number of male patients | 11 |
| Average age (years) | 79.4 ± 3.9 |
| Height (cm) | 153.4 ± 5.8 |
| Weight (kg) | 47.8 ± 5.1 |
| Application to PEG (CVA/CNSD) | 12/7 |
| Nutritional requirements (kcal) per day | 900 ± 0 |
| Period of nutrition supply through a gastric fistula | 16 ± 5 |
| (month) | |
| Development frequency of aspiration pneumonia until | 1.6 ± 0.7 |
| the start time of measurement | |

### <Healthy People>

Tests were performed on 6 healthy people (males; 44 years old to 52 years old; average age: 48 years old). The criteria for exclusion included people who have had an abdominal surgery; and people who were taking medication regularly.

### <Method of Administration to Gastrostomy-Tube Patient>

The test was performed as a random cross test. Each 200 kcal/200 mL of “Elental (registered trade name)” and “Ensure (registered trade name) Liquido” which was labeled using 100 mg of ¹³C sodium acetate (Cambridge Isotope Laboratories, Inc.) was used as a labeled compositional nutrient or a labeled semi-digested nutrient.

Each labeled nutrient was administered to a gastrostomy-tube patient after fasting overnight for exactly 15 minutes at an identical rate (200 mL/15 min) by natural dripping due to gravity. First, any one of the labeled nutrients was administered to one gastrostomy-tube patient, and then, the remaining labeled nutrient was administered to the gastrostomy-tube patient on another day within three days. The analyzer was blind to administration schedule and the order of administering the two types of labeled nutrients was randomly assigned using a sealed opaque envelope.

### <Method of Administration to Healthy Person>

300 kcal/300 mL of “Elental (registered trade name)” and “Ensure (registered trade name) Liquido” which was labeled using 100 mg of ¹³C sodium acetate (Cambridge Isotope Laboratories, Inc.) was used as a labeled compositional nutrient or a labeled semi-digested nutrient.

In the morning after fasting overnight, each labeled nutrient was orally administered to (that is, taken by) a healthy person for 15 minutes, and then, a seated state was maintained over four hours. First, any one of the labeled nutrients was administered to one healthy person, and then, the remaining labeled nutrient was administered to the healthy person on another day within three days.

### <Measurement of Gastric Emptying>

A ¹³C breath test (for example, refer to Shimoyama, et al., Neurogastroenterology and Motility, 2007, vol. 19, pp. 879 to 886) was performed on an expired air sample which was obtained using a closed nasal cannula that was connected to an infrared spectrophotometer (Breath ID System; manufactured by Exalenz Bioscience Ltd.), by measuring a ratio of ¹³CO2 to ¹²CO₂ in each expired air sample, for 4 hours after the administration through a gastric fistula or the oral administration of the labeled nutrient. In this system, exhalations of each subject were automatically collected by automatically measuring a ratio of ¹³CO₂ by infrared emission spectroscopy.

The expired air sample was stored in an intermediate cell which was built into the capnograph in order to control the concentration of carbon dioxide, and then, was subsequently sent to an analysis chamber. Sequence of data during 3h in each subject, consisting of 60-70 measurements, were used for the analysis. The proportion which was obtained from the breath test represents ¹³CO₂ recovery per hour from an initially administered ¹³C ground substance (% dose/h), which corresponds to the gastric emptying rate (% dose/h) at the point of time. The [% dose/h] value is calculated from the ratio of ¹³CO₂ to the ¹²CO₂ in the expired air sample. The [% dose/h] value is calculated from the ratio of ¹³CO₂ to ¹²CO₂ in the expired air sample. The method of using a stable isotope such as ¹³C, in the breath test is advantageous since the method is easily carried out; is noninvasive; and is a quantitative assessment of gastric emission that can be performed with high sensitivity. The method can continuously measure the ratio of ¹³CO₂ to ¹²CO2 in almost real time.

### <Statistical Treatment>

Two way analysis of variance (ANOVA) of repeated measurements was performed to analyze the difference between the compositional nutrient and the semi-digested nutrient groups with respect to cumulative amounts of ¹³CO₂ recovery in the breath at each time point during 50% the three hours after administration in addition to time-effect, in 19 gastrostomy-tube patients. In a case where significant interaction between the treatment and the time was recognized (p value <0.05), a paired comparison was performed after Bonferroni correction. If the p value was smaller than 0.05, it was regarded that there was a significant difference. The statistical analysis was performed using Prism 5 software (GraphPad Software, Inc.).

### <Result>

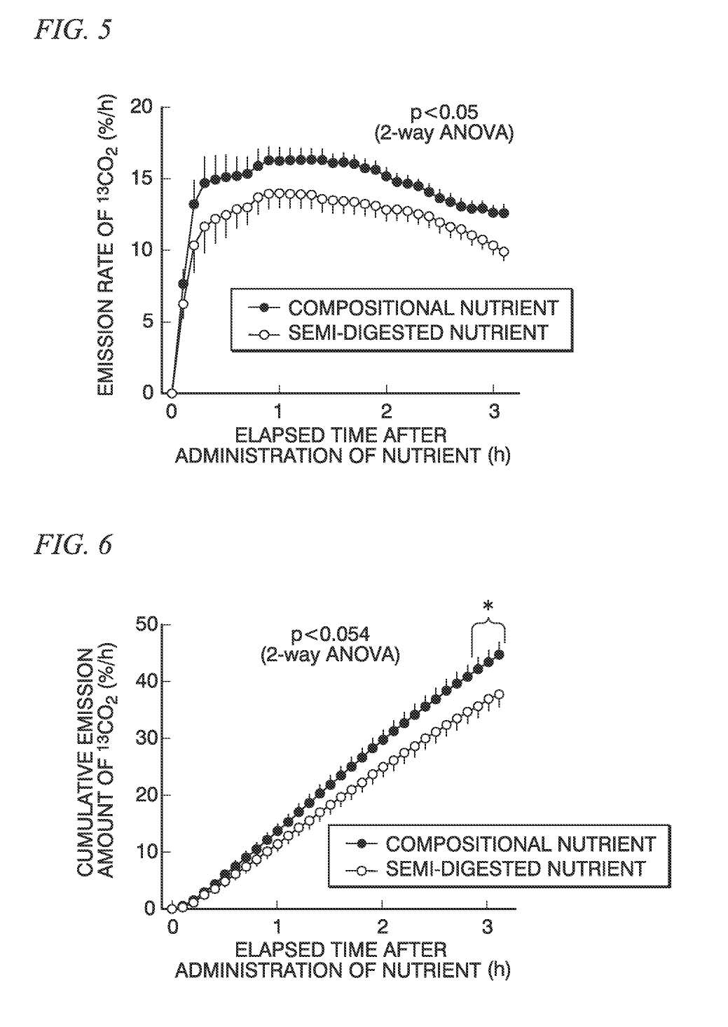

FIG. 5 depicts a curve in which an ¹³CO₂ appearance in the breath ([% dose/h] value) at each point of time after administration of a labeled nutrient to a gastrostomy-tube patient is plotted. FIG. 6 depicts the cumulative ¹³CO₂ recovery in the breath at each point of time after the administration of the labeled nutrient which is calculated using the data in FIG. 5. Furthermore, a result which is obtained by mathematically simulating the times (T₁₀%, T₂₀%, T₃₀%; unit: h) when 10%, 20%, or 30% of total intake of the labeled diet in gastrostomy-tube patients are discharged through exhalations as ¹³CO2 from the curves of the [% dose/h] value in FIG. 5 is shown in Table 5.

TABLE 5

| | When compositional | When semi-digested | |
|---|---|---|---|
| | nutrient was | nutrient was | |
| | administered | administered | p value |
| T_{10%}(h) | 0.82 ± 0.24 | 1.00 ± 0.37 | <0.01 |
| T_{20%}(h) | 1.43 ± 0.34 | 1.72 ± 0.54 | <0.01 |
| T_{30%}(h) | 2.06 ± 0.42 | 2.53 ± 0.76 | <0.01 |

As shown in FIG. 5, after a curve of the [% dose/h] value steeply increases after administration of a labeled compositional nutrient, the curve more moderately increases and reaches a peak, and then, gradually decreases. Furthermore, as shown also in Table 5, with respect to a bedridden gastrostomy-tube patient, the area under the curve of the [% dose/h] value of the labeled compositional nutrient is more significantly increased (p <0.05) compared to that of the labeled semi-digested nutrient. In addition, 10%, 20%, or 30% of the gastric emission time (T₁₀%, T₂₀%, T₃₀%), which is mathematically simulated, is significantly increased (p<0.001, paired t-test). That is, it was confirmed that the compositional nutrient was discharged quicker than the semi-digested nutrient from the stomach to the duodenum.

In a case where the nutritional composition for gastrostomy-tube patients according to the present invention in which a protein source consists of only amino acids is administered through a gastric fistula, the discharge rate of the nutritional composition from the stomach is higher than that of the semi-digested nutrient in the related art, and therefore, aspiration pneumonia rarely develops. Furthermore, the content of lipid, of which the discharge rate from the stomach is comparatively low, in ELENTAL is extremely low, and this point is inferred as one of the causes of marked suppression of the development of aspiration pneumonia in Example 1.

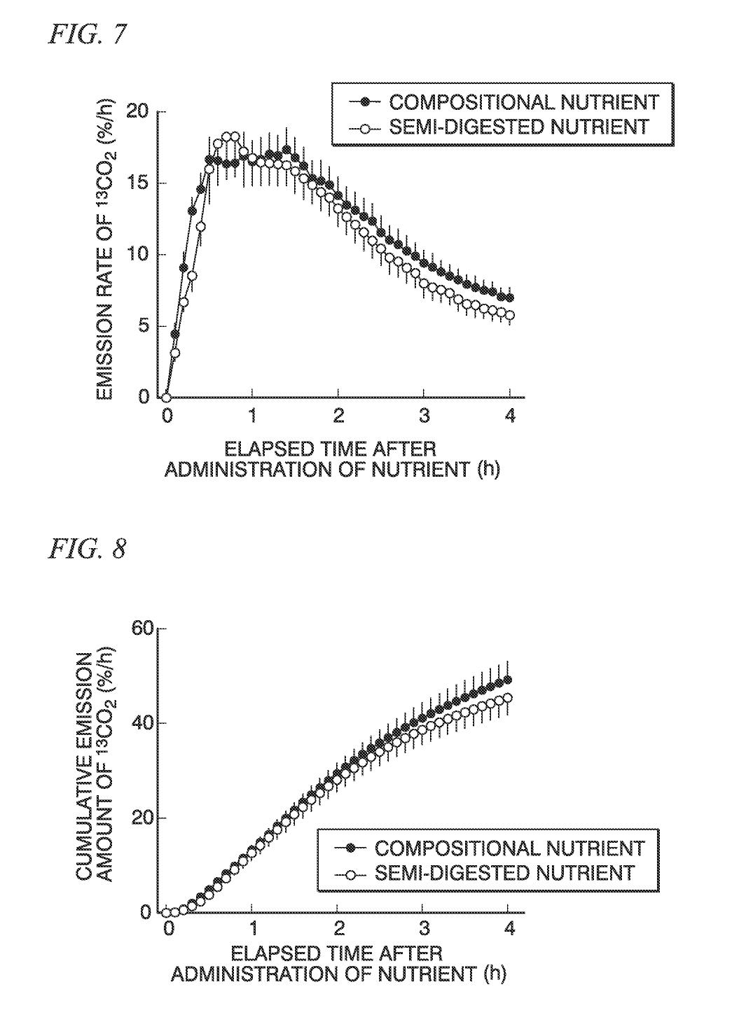

FIG. 7 is a view showing a curve in which an appearance rate of ¹³CO₂ in the breath ([% dose/h] value) at each point of time after administration of a labeled nutrient to a healthy person is plotted. FIG. 8 is a view showing a cumulative of ¹³CO₂ recovery in the breath at each point of time after the administration of the labeled nutrient which is calculated using the data of FIG. 7. Furthermore, a result which is obtained by mathematically simulating the time (T₁₀%, T₂₀%, T₃₀%; unit: h) when 10%, 20%, or 30% of total intake of the labeled diet is discharged through exhalations as ¹³CO₂ from the curves of the [% dose/h] value in FIG. 7 is shown in Table 6.

TABLE 6

| | When compositional | When semi-digested | |
|---|---|---|---|
| | nutrient was | nutrient was | |
| | administered | administered | p value |
| T_{10%}(h) | 0.82 ± 0.09 | 0.90 ± 0.20 | 0.40 |
| T_{20%}(h) | 1.43 ± 0.21 | 1.50 ± 0.33 | 0.67 |
| T_{30%}(h) | 2.14 ± 0.44 | 2.31 ± 0.67 | 0.61 |

As shown in FIGS. 7 and 8 and Table 6, no significant difference in the time (T₁₀%, T₂₀%, T₃₀%) when 10%, 20%, or 30% of each labeled nutrient was discharged from the stomach, and in the area under the curve the [% dose/h] value was observed in the healthy people to which each labeled nutrient was orally administered, in a case where the labeled compositional nutrient was administered and in a case where the labeled semi-digested nutrient was administered.

### Example 3

The multi-chamber container 1A shown in FIG. 1 which was a bag body, of which the plane direction was substantially rectangular; and of which the inside was divided into two chambers; and in which an easily peeled seal, which was formed of a transparent multilayer polyethylene film, was disposed as a communication portion that allowed communication between both the chambers in use, was manufactured as a well-known manufactured device. The outer circumference of the multi-chamber container 1A was unpeelably attached; the peripheral portion on a short side of the bag body of the water injection chamber 2 was provided with the hanging hole 8 and the water injection port 5; and the peripheral portion on a short side of the bag body of the medicine storage chamber 3a was provided with the discharge port 6. The water injection chamber 2 had a cavity which could be filled with 700 mL to 1000 mL of a solution, and the medicine storage chamber 3a stored 240 g of a powdered nutritional composition ELENTAL (manufactured by Ajinomoto Pharmaceuticals Co., Ltd.). The capacity of the medicine storage chamber 3a was 620 mL.

The partition wall portion 4a allowed communication such that 700 mL to 800 mL of water was injected into the multi-chamber container which was obtained in this manner through the water injection port 5, and then, the outside of the water injection chamber 2 was pressed in a state where the water injection port 5 was resealed; and a nutritional liquid composition which could be administered through a tube could be prepared by dissolving the powdered composition which was stored in the medicine storage chamber 3a. The nutritional composition could be administered to a gastric fistula from the discharge port 6 through a tube.

### Example 4

A powdered composition which contained 190.23 g of maltodextrin and 5.796 g of L-glutamine was stored in the medicine storage chamber 3a of the multi-chamber container which was manufactured similarly to Example 3.

331.27 mL of an amino acid infusion agent AMINIC (manufactured by Ajinomoto Pharmaceuticals Co., Ltd.); 4 mL of a trace element preparation ELEMENMIC injection (manufactured by Ajinomoto Pharmaceuticals Co., Ltd.); 2.5 mL of a solution in which a multivitamin preparation MULTIVITAMIN injection (manufactured by Ajinomoto Pharmaceuticals Co., Ltd.) was dissolved in 5 mL of injection water; 72.73 mL of a fat emulsion INTRAFAT injection10% (manufactured by Nihon Pharmaceutical Co., Ltd.); 7 capsules of oral electrolyte agents SORITA injection-T compounded granules No. 3 (manufactured by Ajinomoto Pharmaceuticals Co., Ltd.); and injection water were sequentially injected into the thus obtained multi-chamber container, which was then stored in the water injection chamber 2.

Next, the partition wall portion 4a allowed communication by pressing the outside of the water injection chamber 2 in a state where the water injection port 5 was resealed, and 1000 mL (1 mL/1 kcal) of a nutritional liquid composition, which could be administered through a tube, having the composition disclosed in Table 7, could be prepared by dissolving the powdered composition which was stored in the medicine storage chamber 3a. The nutritional composition could be administered to a gastric fistula from the discharge port 6 through a tube.

TABLE 7

| | Component name | Content per 1000 mL | |
|---|---|---|---|
| Protein | Amino acid | 40 | g |
| Carbohydrate | Dextrin | 190.23 | g |
| Lipid | Soybean oil | 8.8 | g |
| Minerals | Sodium | 560 | mg |
| | Potassium | 546 | mg |
| | Magnesium | 25.2 | mg |
| | Calcium | 70 | mg |
| | Ion | 3.92 | mg |
| | Copper | 0.64 | mg |
| | Manganese | 0.1 | mg |
| | Zinc | 7.94 | mg |
| | Chlorine | 742 | mg |
| | Iodine | 0.26 | μg |
| | Phosphorus | 105 | mg |
| Vitamins | B1 (Thiamin) | 2500 | μg |
| | B2 (Riboflavin phosphate ester) | 2500 | μg |
| | B6 (Pyridoxine) | 2500 | μg |
| | B12 (Cyanocobalamin) | 5 | μg |
| | Pantothenic acid | 7.5 | mg |
| | Niacin (Nicotinic acid amide) | 20 | mg |
| | Folic acid | 200 | μg |
| | Biotin | 50 | μg |
| | C (Ascorbic acid) | 50 | mg |
| | A (Retinol acetate) | 690 | μg |
| | E (Tocopherol acetate) | 7.5 | mg |
| | D (Ergocalciferol) | 5 | μg |
| | K | 1 | mg |
| Calories | | 1000 | kcal |

### INDUSTRIAL APPLICABILITY

The rate of the nutritional composition for gastrostomy-tube patients according to the present invention being discharged from the stomach is high and the development risk of aspiration pneumonia is extremely low. Therefore, the nutritional composition is extremely favorable in supplying nutrition to gastrostomy-tube patients who require risk management for aspiration pneumonia, such as those who sleep in a bed for greater than or equal to 50% of a day or an elderly person.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

### EXPLANATION OF REFERENCES

1A to 1D: MULTI-CHAMBER CONTAINER

2: WATER INJECTION CHAMBER

3a to 3c: MEDICINE STORAGE CHAMBER

4a to 4c: PARTITION WALL PORTION

5: WATER INJECTION PORT

6: DISCHARGE PORT

7: INJECTION PORT

8: HANGING HOLE.

1. A nutritional composition for gastrostomy-tube patients,
wherein the composition is administered to those patients who require risk management for aspiration pneumonia,
wherein the composition comprises a protein source, a carbohydrate source, and a lipid source, and
wherein the protein source consists essentially of amino acids.

2. The nutritional composition for gastrostomy-tube patients according to claim 1,
wherein said protein source is per dry weight:
2 W/W % to 1.5 W/W % of L-isoleucine,
0.5 W/W % to 2.0 W/W % of L-leucine,
0.5 W/W % to 2.0 W/W % of L-lysine,
0.2 W/W % to 1.5 W/W % of L-methionine,
0.5 W/W % to 2.0 W/W % of L-phenylalanine,
0.2 W/W % to 1.5 W/W % of L-threonine,
0.05 W/W % to 0.5 W/W % of L-tryptophan,
0.2 W/W % to 1.5 W/W % of L-valine,
0.5 W/W % to 2.0 W/W % of L-histidine,
0.5 W/W % to 2.5 W/W % of L-arginine,
0.5 W/W % to 2.0 W/W % of L-alanine,
1.0 W/W % to 4.0 W/W % of L-aspartic acid,
1.0 W/W % to 4.0 W/W % of L-glutamine,
0.2 W/W % to 1.5 W/W % of glycine,
0.2 W/W % to 1.5 W/W % of L-proline,
0.5 W/W % to 2.5 W/W % of L-serine, and
0.05 W/W % to 0.5 W/W % of L-tyrosine.

3. The nutritional composition for gastrostomy-tube patients according to claim 1,
wherein the carbohydrate source is 70 W/W % to 85 W/W % of dextrin per dry weight, and
wherein the lipid source is 0.1 W/W % to 10 W/W % of soybean oil per dry weight.

4. The nutritional composition for gastrostomy-tube patients according to claim 3,
wherein the lipid source is 0.3 W/W % to 1.0 W/W % per dry weight.

5. The nutritional composition for gastrostomy-tube patients according to claim 1,
wherein the composition is administered such that the dose per day is greater than or equal to 900 kcal.

6. The nutritional composition for gastrostomy-tube patients according to claim 1,
wherein the composition can be continuously administered over 20 months or longer while suppressing the incidence of aspiration pneumonia to be less than or equal to 3%.

7. A device useful for preparing a nutritional composition for gastrostomy-tube patients, the device comprising:
a composition comprising at least one source selected from the group consisting of a protein source, a carbohydrate source, and a lipid source;
a multi-chamber container having at least two chambers and a communicating partition wall portion formed of a flexible film partitioning the at least two chambers, the composition being stored in said container;
wherein one of the at least two chambers is a water injection chamber and has a storage capacity greater than or equal to 200 mL of a liquid, and further comprises a resealable water injection port through which liquid can be injected and discharged;
wherein at least one of the at least two chambers comprises at least one medicine storage chamber, in which at least a part of said composition is stored; and
wherein, in at least one of the medicine storage chambers, at least a part of the composition is stored in a solid form.

8. The device according to claim 7,
wherein the partition wall portion is configured and arranged to allow communication between the water injection chamber and at least one medicine storage chamber, such that the nutritional composition for gastrostomy-tube patients can be prepared in the sealed multi-chamber container by pressing the water injection chamber from outside the chamber when the water injection port is sealed after injecting water into the water injection chamber through the water injection port.

9. The device according to claim 7,
wherein the multi-chamber container further comprises a discharge port configured and arranged to allow communication with a tube that is connectable to a gastric fistula.

10. The device according to claim 7, wherein:
one of the at least two chambers is a first medicine storage chamber in which a part of said composition is stored in a solid form, and a second of the at least two chambers is a second medicine storage chamber in which the remainder of the composition is stored; and
wherein at least a carbohydrate source is stored in the first medicine storage chamber.

11. The device according to claim 10, wherein L-glutamine is stored in the first medicine storage chamber.

12. The device according to claim 10,
wherein at least one composition selected from the group consisting of a fat emulsion, an antiflatulent, and a medicine for treating a disease, are stored in the second medicine storage chamber.

13. The device according to any one of claims 7 to 12, wherein:
the multi-chamber container further comprises a pipe member having a cylindrical space and a rubber plug that blocks the cylindrical space;
the cylindrical space allows communication between the multi-chamber container and outside the device; and
the rubber plug can be penetrated by a hollow needle and can be resealed by withdrawing the hollow needle.

14. The device according to claim 7, further comprising
an inert gas is substituted in the water injection chamber.

15. The device according to claim 7,
wherein the multi-chamber container is sterilized by radiation.

16. The device according to claim 7, further comprising:
an external packaging bag and a deoxygenating agent, a drying agent, or both, therein;
wherein the multi-chamber container is air-tightly stored in said external packaging bag.

17. The device according to claim 16,
wherein the external packaging bag is formed of a flexible film which has light-shielding properties and through which gas does not permeate.

18. The device according to claim 7,
wherein the nutritional composition for gastrostomy-tube patients contains only amino acids as the protein source.

19. A device useful for preparing a nutritional composition for gastrostomy-tube patients, the device comprising:
a nutritional composition for gastrostomy-tube patients according to claim 1;
a multi-chamber container having at least two chambers and a communicating partition wall portion formed of a flexible film partitioning the at least two chambers, the composition being stored in said container;
wherein one of the at least two chambers is a water injection chamber and has a storage capacity greater than or equal to 200 mL of a liquid, and further comprises a resealable water injection port through which liquid can be injected and discharged;
wherein at least one of the at least two chambers comprises at least one medicine storage chamber, in which at least a part of said composition is stored in a solid form.

20. A method of manufacturing a device useful for preparing a nutritional composition for gastrostomy-tube patients, the method comprising:
providing a flexible film, a fluid port device, and a composition according to claim 1;
positioning said composition adjacent to said flexible film;
positioning said fluid port device at an edge of said flexible film; and
sealing the flexible film to form a container having at least two compartments and a partition wall releasably separating the at least two compartments, said sealing including sealing said fluid port device at said edge such that the fluid port device fluidly communicates one of said compartments with the exterior of said container, and said composition is sealed in another of said at least two compartments.
